(19) United States
(12) Patent Application Publication
(10) Pub. No.: US 2012/0291535 A1
(43) Pub. Date: Nov. 22, 2012

(54) OIL MIST DETECTOR TEST RIG

(75) Inventors: Ronald Paul Maloney, Peoria, IL (US); Eric Nathan Lucas, Washington, IL (US); Joseph George Chambers, Chillicothe, IL (US)

(73) Assignee: Caterpillar Inc., Peoria, IL (US)

(21) Appl. No.: 13/112,298

(22) Filed: May 20, 2011

(51) Int. Cl. G01N 33/30

(52) U.S. Cl. 73/114.57; 73/114.56

(57) ABSTRACT

An oil mist detector test rig including an oil container, an aerosol generator, an oil mist detector, and a collection unit. The aerosol generator generates fluid aerosol in the oil container. The oil mist detector is configured to provide a first reading based on a concentration of the generated fluid aerosol in the oil container. Additionally, the collection unit is configured to provide a second reading based on the concentration of the generated fluid aerosol in the oil container.

### TECHNICAL FIELD

The present disclosure relates to an oil mist detector, and more particularly to an oil mist detector test rig.

### BACKGROUND

Oil mist detectors are used to detect concentration of oil mist generated in an engine crankcase. Japanese Patent Application Publication Number JP2007278942A (942 application) discloses a device for inspecting the oil mist detector. The '942 application discloses that the oil mist flows into a test vessel from an oil mist generation device. The '942 application further discloses that the test vessel includes an optical oil mist sensor for detecting a concentration of the oil mist in the test vessel, and an oil mist measurement device for measuring the concentration of the oil mist in the test vessel by a weight method.

### SUMMARY OF THE DISCLOSURE

In one aspect, the present disclosure provides an oil mist detector test rig including an oil container, an aerosol generator, an oil mist detector, and a collection unit. The aerosol generator generates fluid aerosol in the oil container. The oil mist detector is configured to provide a first reading based on a concentration of the generated fluid aerosol in the oil container. Also, the collection unit is configured to provide a second reading based on the concentration of the generated fluid aerosol in the oil container.

In another aspect, the present disclosure provides a method for testing an oil mist detector in an oil mist detector test rig. The method generates fluid aerosol in an oil container. The method then obtains, from the oil mist detector, a first reading based on a concentration of the generated fluid aerosol. Further, the method obtains, from a collection unit, a second reading based on the concentration of the generated fluid aerosol in the oil container. Subsequently, the method compares the first reading and the second reading, in order to test one or more parameters of the oil mist detector. In one aspect, the method may calibrate the one or more parameters of the oil mist detector based, at least in part, on the compared readings.

In yet another aspect, a test rig is provided for testing an oil mist detector. The test rig includes a simulated crankcase of an internal combustion engine, an oil mist detector, and a collection unit. The simulated crankcase further comprises an oil container, a pressurized air source, one or more nozzles and at least one gas sampling manifold. The pressurized air source is used to generate fluid aerosol in the oil container. Further, the one or more nozzles spray liquid fluid particles in the oil container, such that the liquid fluid particles are suspended with the generated fluid aerosol. Further, the at least one gas sampling manifold is associated with the oil container and is configured to receive the generated fluid aerosol with the suspended liquid fluid particles. The oil mist detector which is to be tested is configured to provide a first reading based on a concentration of the generated fluid aerosol in the oil container. Also, the collection unit is configured to provide a second reading based on the concentration of the generated fluid aerosol in the oil container.

Other features and aspects of this disclosure will be apparent from the following description and the accompanying drawings.

### BRIEF DESCRIPTION OF THE DRAWINGS

FIG. 1 is a schematic view of an oil mist detector test rig.

FIG. 2 is a detailed schematic view of the oil mist detector test rig setup.

FIG. 3 is a perspective view of an oil container and gas sampling manifolds.

FIG. 4 is a block diagram of a testing sequence.

### DETAILED DESCRIPTION

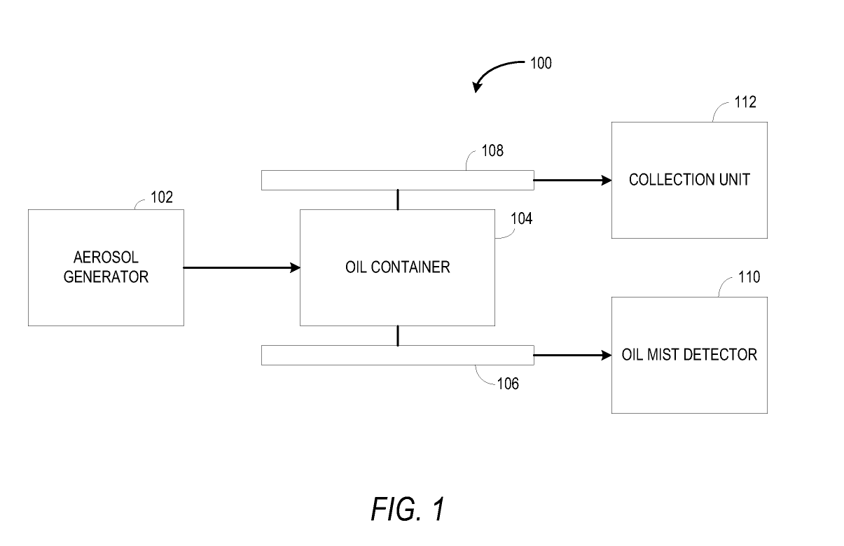

FIG. 1 is a schematic of an oil mist detector test rig 100 (herein after interchangeably referred to as test rig 100). The test rig 100 may include an aerosol generator 102, an oil container 104, at least one gas sampling manifold 106, 108, an oil mist detector 110, and a collection unit 112.

In an embodiment of the present disclosure, the aerosol generator 102, the oil container 104, and the gas sampling manifolds 106, 108 may simulate a crankcase of an internal combustion engine. The oil container 104 may include low viscosity fluid. The low-viscosity fluid may be used instead of engine oil to avoid possible combustible environment in the oil container 104. At room temperature or under test conditions, the low viscosity fluid may replicate oil that may be contained in a real engine crankcase under given in-use conditions. Therefore the fluid will be referred to as oil in this application. However, it may be apparent to a person of ordinary skill in the art that the oil container 104 may include any kind of fluid that may simulate the engine oil.

Further, a sheet metal box may be utilized as the oil container 104 to simulate a body of the crankcase. However, it may be apparent to a person of ordinary skill in the art that any kind of suitable material and structure may be used to replicate the body of the crankcase.

The aerosol generator 102 is associated with the oil container 104. The aerosol generator 102 may be provided to agitate the oil in the oil container 104 and thus may produce oil aerosol. The generated oil aerosol may replicate an actual oil mist that may be produced in the crankcase of the engine.

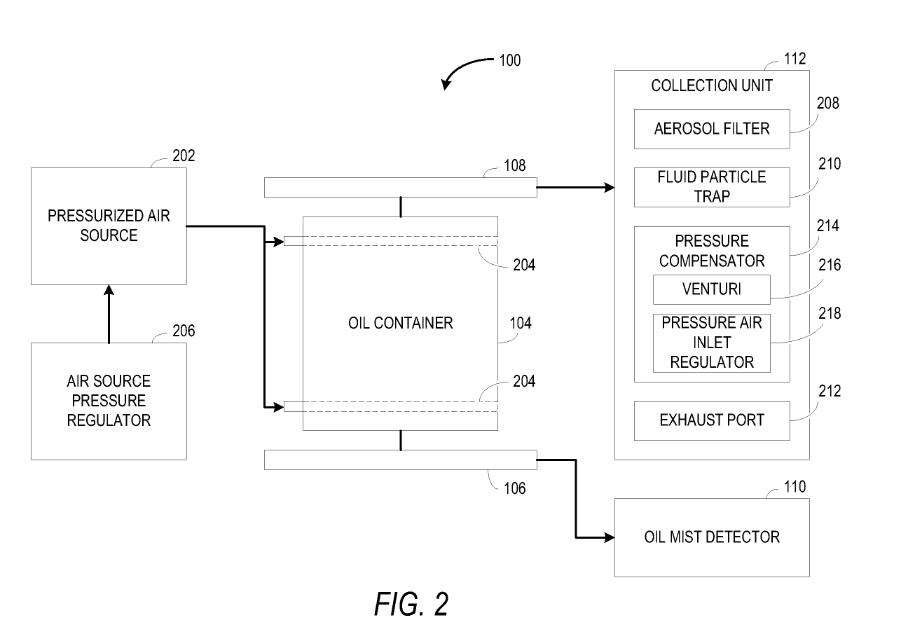

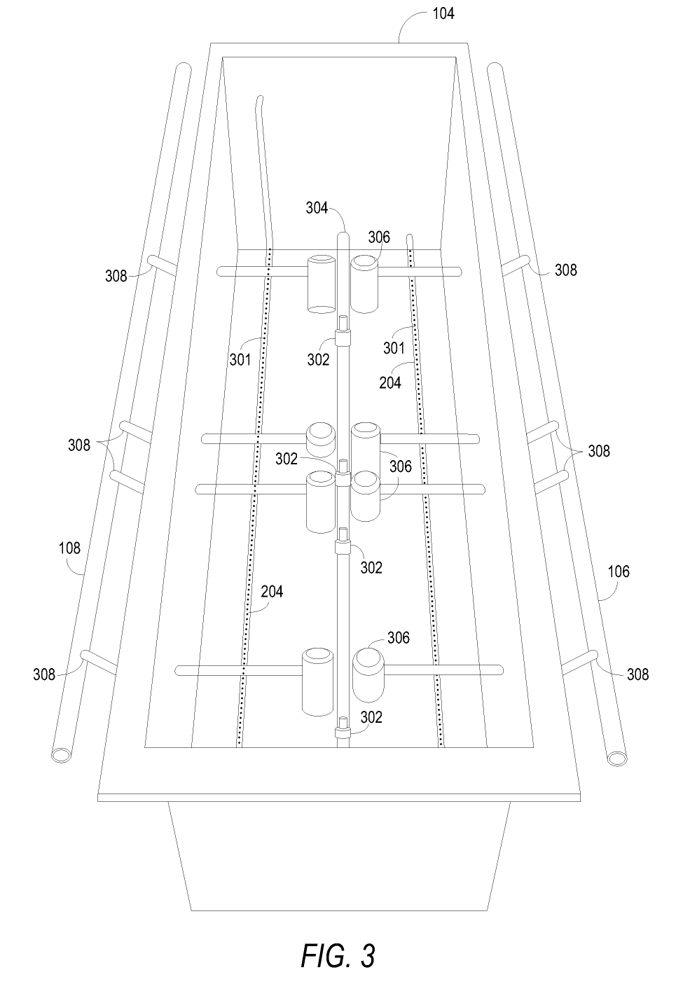

As shown in FIG. 2, the aerosol generator 102 may include a pressurized air source 202 such as, but not limited to, an air compressor. The pressurized air source 202 may supply pressurized air to at least one submerged air lines 204 placed inside the oil container 104. As shown in FIG. 3, the submerged air lines 204 may be present in the inner lower and/or side walls of the oil container 104. Pressurized air may flow through the submerged air lines 204 and may escape out of one or more apertures 301 provided in the submerged air lines 204. The pressurized air coming out of the one or more apertures 301 may agitate the fluid and thus generate the fluid aerosol in the oil container 104.

Further, an air source pressure regulator 206 may be associated with the pressurized air source 202. The air source pressure regulator 206 may vary the pressure of the pressurized air source 202 to change the concentration of the generated fluid aerosol inside the oil container 104. This may simulate one or more operating conditions of engine, such as, but not limited to, an engine idle condition, a steady engine condition, a maximum load engine condition, and the like. An increase in the pressure associated with the pressurized air source 202 may cause a corresponding increase in the concentration of the generated fluid aerosol in the oil container 104.

In another embodiment, the aerosol generator 102 may include high pressure injectors. It is apparent to a person ordinarily skilled in the art that the above methods of aerosol generation are for exemplary purposes only and any known in the art process can be used to produce the fluid aerosol in the oil container 104 without deviating from the scope of the disclosure.

As shown in the FIG. 3, one or more nozzles 302 may be placed along a central axis of the oil container 104. The nozzles 302 may spray the liquid fluid particles in the oil container 104. The spraying of the liquid fluid particles in the oil container 104 may replicate liquid oil throw off from the rotating components, such as piston, in the crankcase of the engine. The liquid fluid particles sprayed from the nozzles 302 may get suspended with the fluid aerosol generated by the pressurized air source 202. In an embodiment, the one or more nozzles 302 may receive the fluid from the oil container 104 through a supply line 304. Alternatively, the nozzles 302 may receive the fluid from an external oil tank through the supply line 304.

Further, the supply line 304 may also include a pressure regulator (not shown) to vary the pressure of the fluid reaching the nozzles 302. The variation in the pressure of the fluid reaching the nozzles 302 may vary the quantity of liquid fluid spraying in the oil container 104 at any given instant of time. The variation in the quantity of the liquid fluid particle sprayed in the oil container 104 may simulate the liquid oil throw off by the rotating parts of the engine at varying load conditions of the engine.

Specifically, the nozzles 302 may impinge the liquid fluid particles on one or more suction bells 306 placed adjacent to the central axis of the oil container 104. The suction bells 306 may be configured to be fluidly connected with the oil container 104 and to draw out the fluid aerosol with the suspended liquid fluid particles from the oil container 104. The suction bells 306 are connected to one or more sampling ports 308, which may be fluidly connected with the suction bells 306. In an embodiment, the sampling ports 308 may be appropriately positioned inside the oil container 104 to receive the fluid aerosol from all portions of the oil container 104.

Specifically, the sampling ports 308 may be placed at an end of tubes extending from the suction bells 306 and on both sides of the oil container 104. The sampling ports 308 may provide the fluid aerosol with the suspended liquid fluid particles to the gas sampling manifolds 106, 108. In an embodiment of the present disclosure, the gas sampling manifolds 106, 108 may be tubes fluidly connected to the oil container 104 as shown or could be integrally formed with the oil container 104. Specifically, the gas sampling manifolds 106, 108 may act as an inlet for the fluid aerosol with the suspended fluid particles from the oil container 104. As shown in the Figures, there may be one gas sampling manifold 106, 108 on each side of the oil container 104. The gas sampling manifolds 106, 108 may be fluidly connected to the sampling ports 308 to receive the fluid aerosol from the oil container 104.

As shown in the FIG. 2, one of the gas sampling manifolds 106 may be connected with an inlet of the oil mist detector 110 and the other gas sampling manifold 108 may be connected with an inlet of the collection unit 112. The gas sampling manifolds 106, 108 may be connected to the oil mist detector 110 and the collection unit 112 by any suitable means such as, but not limited to, hoses. The connection means may further include instruments such as, but not limited to, a flow regulator, a flow meter, and the like.

In an embodiment of the present disclosure, an approximately equal quantity of oil aerosol containing the suspended liquid fluid particles may be provided from the gas sampling manifolds 106, 108 to the oil mist detector 110 and the collection unit 112 respectively. Specifically, the flow regulator provided with the connection means may ascertain that approximately equal quantity of fluid aerosol containing the suspended liquid fluid particles may be provided from the gas sampling manifolds 106, 108 to the oil mist detector 110 and the collection unit 112 respectively. In an embodiment of the present disclosure, the oil mist detector 110 may provide the first reading based on the concentration of the generated fluid aerosol in the oil container 104, from the oil mist detector 110. The oil mist detector 110 may have an aerosol concentration reader coupled with the oil mist detector 110 to provide the first reading.

Further, the collection unit 112 may provide the second reading based on the concentration of fluid aerosol in the oil container 104, from the collection unit 112. The collection unit 112 may include an aerosol filter 208 and a fluid particle trap 210. An inlet of the collection unit 112 may receive the fluid aerosol with suspended liquid fluid particles from the outlet of the gas sampling manifold 108. The suspended liquid fluid particles may get trapped in the fluid particle trap 210 provided in the collection unit 112. It may be apparent to a person of ordinary skill in the art that the fluid particle trap 210 may be any known instrument in the art such as, but not limited to, a breather. Subsequently, the fluid aerosol may pass through the aerosol filter 208. The aerosol filter 208 may trap the small liquid fluid particles from the oil aerosol. The rest of the gases (segregated of fluid aerosol and suspended liquid fluid particles) may exhaust out from an exhaust port 212 provided in the collection unit 112.

Further, the collection unit 112 may also include a pressure gauge to provide a pressure reading of the fluid aerosol (segregated of the suspended liquid fluid particles). The pressure reading may indicate a pressure drop in the fluid aerosol (with the suspended liquid fluid particles) from the inlet of the collection unit 112 to the fluid aerosol (segregated of the suspended liquid fluid particles) coming out of the fluid particle trap 210. In an embodiment of the present disclosure, the collection unit 112 may also include a pressure compensator 214. The pressure compensator 214 may include a venturi 216 and a pressure air inlet regulator 218. The venturi 216 and the pressure air inlet regulator 218 may compensate any pressure loses from the fluid aerosol as it passes through the fluid particle trap 210. The collection unit 112 may provide the second reading based on a weight of the fluid aerosol collected by the aerosol filter 208.

In an embodiment of the present disclosure, the second reading may be taken manually by taking out the aerosol filter 208 from the collection unit 112. The second reading may be provided based on an approximate weight of the fluid aerosol that may be collected by the aerosol filter 208. However, it may be apparent to a person of ordinary skill in the art that the exemplary method stated above is merely on an illustrative basis. Any other known manual and/or automated method may be used to estimate the concentration of fluid aerosol from the aerosol filter 208 provided in the collection unit 112.

In an embodiment of the present disclosure, the test rig 100 may also include the comparator unit (not shown in the Figures). The comparator unit may compare the first reading, obtained from the oil mist detector 110 and the second reading obtained from the collection unit 112. Further, the comparator unit may calibrate the one or more parameters such as, but not limited to, sensitivity of the oil mist detector 110 based on the compared readings.

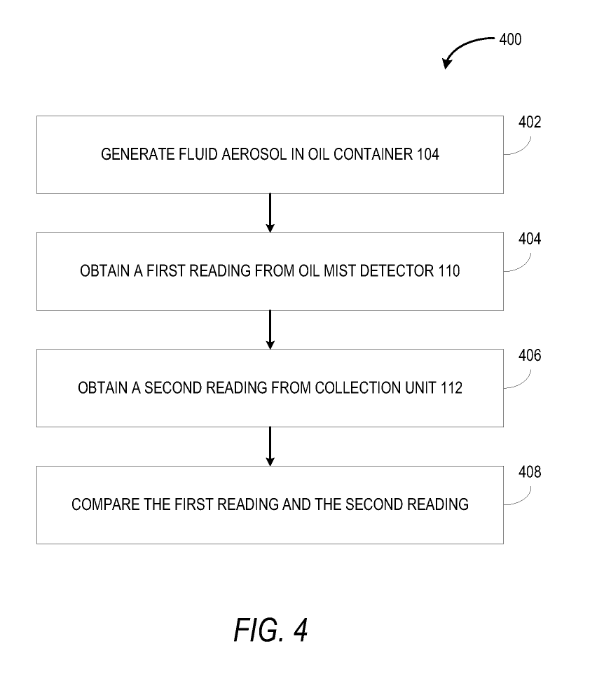

FIG. 4 illustrates a testing sequence 400 in the test rig 100. In step 402, the aerosol generator 102 may generate fluid aerosol in the oil container 104. The generated fluid aerosol may replicate the actual oil mist that may be produced in the crankcase of the engine.

Further, the test rig 100 may also include the nozzles 302 placed in the oil container 104. The nozzles 302 may be configured to spray liquid fluid particles in the oil container 104. The spraying of the liquid fluid particles in the oil container 104 may replicate liquid oil throw off from the rotating components, such as piston, in the crankcase of the engine. The liquid fluid particles sprayed from the nozzles 302 may get suspended with the fluid aerosol generated by the pressurized air source 202.

Further, in an embodiment, the pressure of at least one of the pressurized air source 202 and/or the nozzles 302 may be altered to vary the concentration of fluid aerosol containing the suspended liquid fluid particles in the oil container 104. The varying levels of fluid aerosol concentration in the oil container 104 may simulate the actual change in oil aerosol concentration in the crankcase corresponding to varying load conditions in the engine.

In step 404, the first reading may be obtained from the oil mist detector 110 based on the generated fluid aerosol in the oil container 104. In an embodiment of the present disclosure, gas sampling manifolds 106, 108 are coupled with the oil container 104 to receive the fluid aerosol from the oil container 104. The gas sampling manifolds 106, 108 may receive the fluid aerosol from the oil container 104 through the sampling ports 308.

The gas sampling manifold 106 may provide a predetermined volume of the generated fluid aerosol with suspended liquid fluid particles to the oil mist detector 110 based on which the first reading is obtained from the oil mist detector 110.

In step 406, the second reading is obtained from the collection unit 112 based on the concentration of fluid aerosol in the oil container 104. The collection unit 112 may receive the approximately equal amount of fluid aerosol from the gas sampling manifold 108 as that of the oil mist detector 110. The collection unit 112 may include the aerosol filter 208 and the fluid particle trap 210, to provide the second reading corresponding to the fluid aerosol generated in the oil container 104.

In an embodiment of the present disclosure, various readings may be obtained from the oil mist detector 110 and the collection unit 112 corresponding to the varying level of fluid aerosol generated in the oil container 104.

In step 408, the first reading, obtained from the oil mist detector 110, and the second reading, obtained from the collection unit 112, is compared to test the one or more parameters of the oil mist detector 110. In an embodiment of the present disclosure, the test rig 100 may also include the comparator unit. The comparator unit may compare the first and the second reading obtained from the oil mist detector 110 and the collection unit 112 respectively. Subsequently, the comparator unit may calibrate the one or more parameters of the oil mist detector 110 based on the compared readings. The one or more parameters may include, but not limited to, sensitivity of the oil mist detector 110.

### INDUSTRIAL APPLICABILITY

The oil mist detector 110 is generally used with engines, where moving components of the engines may produce oil mist inside the crankcase. The high level of oil mist may cause an explosion in the crankcase of the engine. The oil mist detector 110 may produce an alarm if the generated oil mist in the crankcase reaches beyond a pre-defined limit. However, if the sensitivity of providing reading and thus producing an alarm, of the oil mist detector 110 is not calibrated accurately, then the oil mist detector 110 may often produce false alarms.

The test rig 100 described above, may be used to test any known in the art oil mist detector 110. The one or more parameters of the oil mist detector 110 may be tested using the test rig 100. The test rig 100 may provide a compact as well as efficient technique of simulating the one or more operating conditions of the engine crankcase, including the idle engine, overload condition, etc. Thus, the test rig 100 may allow testing of the oil mist detector 110 in variety of engine conditions without the costs and complication of running an actual engine. The test rig 100 may also provide more repeatable and controllable conditions than a real engine could easily do.

Oil aerosol including suspended liquid oil particles (herein after interchangeably referred to as oil mist) may be created in the crankcase of an engine when lubricating oil may be splashed by moving and rotating parts of the engine. The oil mist may reduce the flash point of the oil, allowing it to catch fire in presence of a hot spot. Thus, it is important that the concentration of oil aerosol is kept under control and incase its presence is detected, the engine should be stopped or the speed lowered.

For this purpose, the oil mist detector 110 may be used in conjunction with the crankcase of the engine. If the amount of oil mist increases, the oil mist detector 110 may raise an alarm. The oil mist detector 110 may not reduce or prevent the formation of oil mist, but may give a warning in case the concentration may rise above a given level.

Presently, the one or more parameters of the oil mist detector 110 may be calibrated, based on the type of the engine the oil mist detector 110 needs to be used with. Further, the sensitivity of the oil mist detector 110 may also be checked to avoid any false alarms. Typically, such tests and calibration of the oil mist detector 110 are carried out on the real engine crankcase, which may not be desirable considering size of the engine, fuel burn, cost of test cell operation, and the like. However the proposed test rig 100 explained above may be used to simulate a real engine crankcase. Further, various operating conditions of the engine crankcase may be simulated by varying the concentration of the oil mist that may be produced in the oil container 104. The air source pressure regulator 206 may be used to vary the pressure of the pressurized air source 202 and thus, vary the concentration of the oil mist in the oil container 104.

Further, the collection unit 112 of the test rig 100 may be used to compare the readings that may be obtained from the oil mist detector 110, for the generated oil mist in the oil container 104 under various simulated operating conditions of the engine. Specifically, the reading obtained from the collection unit 112 for approximately the same quantity of the oil mist that have been provided to the oil mist detector 110; may be compared with the reading obtained from the oil mist detector 110. Based, on the compared readings, the one or more parameters of the oil mist detector 110 may be calibrated.

The calibration of the oil mist detector 110 to suit to the specific engine type, under varying load conditions may prevent false alarms. Further, the calibration is carried out on the simulated crankcase of the engine which may be desirable from time and cost perspective. Additionally, the compactness of the design of the test rig 100 may provide a testing setup that may be easy to transport and/or configure. It is apparent to a person who is ordinary skilled in the art that the above test rig 100 can be used for testing or calibrating any known in the art oil mist detector 110.

Although the embodiments of this disclosure as described herein may be incorporated without departing from the scope of the following claims, it will be apparent to those skilled in the art that various modifications and variations can be made. Other embodiments will be apparent to those skilled in the art from consideration of the specification and practice of the disclosure. It is intended that the specification and examples be considered as exemplary only, with a true scope being indicated by the following claims and their equivalents.

What is claimed is:

1. An oil mist detector test rig comprising:
an oil container;
an aerosol generator configured to generate fluid aerosol in the oil container;
an oil mist detector configured to provide a first reading based on a concentration of fluid aerosol in the oil container; and
a collection unit configured to provide a second reading based on the concentration of fluid aerosol in the oil container.

2. The oil mist detector test rig of claim 1, wherein the aerosol generator includes a pressurized air source.

3. The oil mist detector test rig of claim 2, wherein the pressurized air source further includes submerged air lines.

4. The oil mist detector test rig of claim 1, wherein the aerosol generator includes a nozzle placed along a central axis of the oil container and wherein the nozzle is configured to spray liquid fluid particles in the oil container.

5. The oil mist detector test rig of claim 1 further includes a gas sampling manifold in fluid communication with the oil container.

6. The oil mist detector test rig of claim 1 further includes one or more suction bells placed adjacent to a central axis of the oil container, and wherein the suction bells is configured to provide the fluid aerosol from the oil container to at least one sampling port.

7. The oil mist detector test rig of claim 6, wherein the sampling port is placed at an end of tubes extending form the suction bells, and wherein the sampling port is in fluid communication with the gas sampling manifold.

8. The oil mist detector rig of claim 1, wherein the oil container contains low viscosity fluid to simulate engine oil.

9. The oil mist detector rig of claim 1, wherein the collection unit includes an aerosol filter.

10. The oil mist detector rig of claim 1, wherein the collection unit includes a fluid particle trap.

11. The oil mist detector rig of claim 1, wherein the collection unit includes a pressure regulator.

12. The oil mist detector rig of claim 1 further includes a pressure regulator associated with the aerosol generator.

13. A method for testing an oil mist detector in an oil mist detector test rig, the method comprising:
generating fluid aerosol in an oil container;
obtaining, from the oil mist detector, a first reading based on a concentration of the generated fluid aerosol;
obtaining, from a collection unit, a second reading based on the concentration of the generated fluid aerosol; and
comparing the first reading and the second reading to test one or more parameters of the oil mist detector.

14. The method of claim 13 further includes calibrating the one or more parameters of the oil mist detector based, at least in part, on the compared readings.

15. The method of claim 13, wherein generating fluid aerosol includes providing compressed air in the oil container.

16. The method of claim 13, wherein generating fluid aerosol includes spraying liquid fluid particles in the oil container.

17. The method of claim 13 further includes varying the concentration of the fluid aerosol in the oil container.

18. A test rig comprising:
a simulated crankcase of an internal combustion engine, the simulated crankcase including:
an oil container;
a pressurized air source to generate fluid aerosol in the oil container;
one or more nozzles to spray liquid fluid particles in the oil container, wherein the fluid particles are suspended with the generated fluid aerosol; and
at least one gas sampling manifold associated with the oil container, wherein the at least one gas sampling manifold is configured to receive the generated fluid aerosol with the suspended low viscosity fluid particles;
an oil mist detector, to be tested, configured to provide a first reading based on a concentration of the generated fluid aerosol in the oil container; and
a collection unit configured to provide a second reading based on the concentration of the generated fluid aerosol in the oil container.

19. The test rig of claim 18, wherein the pressurized air source further includes submerged air lines.

20. The test rig of claim 18 further includes a pressure regulator associated with the simulated crankcase, wherein the pressure regulator is configured to vary the concentration of the generated fluid aerosol to simulate one or more operating conditions of the engine.
